(19) United States
(12) Patent Application Publication
(10) Pub. No.: US 2026/0354718 A1
(43) Pub. Date: Nov. 12, 2026

(54) TIME AND SPATIALLY RESOLVED HYPERSPECTRAL IMAGING OF DEEP TISSUE OPTICAL PROPERTIES, HEMODYNAMICS, AND METABOLISM

(71) Applicant: University of Kentucky Research Foundation, Lexington, KY (US)

(72) Inventors: Guoqiang Yu, Lexington, KY (US); Faraneh Fathi, Lexington, KY (US); Faezeh Akbari, Lexington, KY (US); Lei Chen, Lexington, KY (US)

(21) Appl. No.: 19/647,340

(22) Filed: Apr. 14, 2026

Related U.S. Application Data
(60) Provisional application No. 63/788,435, filed on Apr. 14, 2025.

(51) Int. Cl. A61B 5/1455; A61B 5/00; A61B 5/0205; A61B 5/026; H04N 23/12

(52) U.S. Cl. CPC A61B 5/14552; A61B 5/0205; A61B 5/0261; A61B 5/4866; A61B 5/7278; H04N 23/12; A61B 2576/00

(57) ABSTRACT

A time- and spatially-resolved hyperspectral imaging (TSR-HSI) system. The system includes a wavelength-tunable pulsed laser configured to sequentially emit illumination at a plurality of wavelengths, a coherent pulsed laser having a spectral bandwidth less than 1 nm, and a gated SPAD camera configured for time-resolved photon detection. The system also includes a synchronization controller configured to operate the wavelength-tunable pulsed laser as a master timing source and synchronize emission of the coherent pulsed laser and gating of the SPAD camera to the master timing source; and a processor configured to acquire, using the SPAD camera, multispectral intensity data from the plurality of wavelengths and speckle contrast data from the coherent pulsed laser, and determine depth-resolved values of at least two of tissue absorption coefficient, tissue scattering coefficient, blood flow index, and oxygen saturation. The depth resolution is achieved by selecting different temporal gates corresponding to different photon path lengths.

### CROSS-REFERENCE TO RELATED APPLICATIONS

This application claims priority to U.S. Provisional Application 63/788,435, filed Apr. 14, 2025, which is incorporated by reference herein in its entirety.

### TECHNICAL FIELD

Embodiments of the present disclosure generally relate to apparatus and methods for determining deep tissue optical properties, hemodynamics, and metabolism. More particularly, embodiments of the present disclosure relate to time and spatially resolved hyperspectral imaging (TSR-HSI) systems, methods for imaging tissue, and methods for determining optical and physiological parameters.

### BACKGROUND

Blood flow (BF) carries nutrition and oxygen to tissues while also removing metabolic waste products. When oxygen demand exceeds oxygen supply, organ/tissue malfunction or permanent tissue damage may occur depending on the severity and duration of oxygen deprivation. Because BF and tissue blood oxygen saturation (StO₂) may not be coupled, combination of BF and STO₂ measurements enables distinguishing causes of tissue ischemia and tissue hypoxia which occurs in many diseases including cerebral disease, cardiovascular disease, peripheral vascular disease, cancer, diabetes, burn/wound injury, angiogenesis or tissue/vascular reconstruction injury. From BF and STO₂ measurement results, one can derive the metabolic rate of tissue oxygen consumption (TMRO₂), another important functional parameter tightly associated with tissue pathophysiology. Taken together, simultaneous measurements of BF and StO₂ enable the diagnosis of various diseases and the monitoring of related medical interventions.

Currently available neuroimaging technologies for deep tissue hemodynamics/metabolism measurements include magnetic resonance imaging (MRI), positron emission tomography (PET), x-ray computed tomography (CT) and transcranial doppler ultrasound (TCD). While useful, these imaging modalities present a host of issues that restrict their applications. MRI, PET, and CT are large, expensive, and difficult to use for continuous and longitudinal monitoring of tissue hemodynamics. PET uses ionizing radiation and CT involves exposing the patient to a significant amount of radiation, thus limiting their clinical applications. TCD enables the detection of blood flow in large vessels but is not sensitive to microcirculation.

By contrast, optical imaging instruments are portable, inexpensive, and fast, enabling continuous assessment of tissue hemodynamics in the microvasculature at the bedside. Laser speckle contrast imaging (LSCI) and optical intrinsic signal imaging (OISI) with widefield illumination and charge-coupled device/complementary-metal-oxide-semiconductor (CCD/CMOS) camera detection enable fast and high-resolution 2D mapping of cerebral blood flow (CBF) and cerebral blood oxygen saturation (ScO₂) on cortices of rodents, respectively. However, due to their limited penetration depth (<1 mm), LSCI and OISI require the retraction of the animal's scalp and/or an invasive cranial window for cortical imaging. As a result, LSCI and OISI are difficult to use for noninvasive deep brain imaging.

Conventional diffuse optical technologies such as near-infrared spectroscopy (NIRS) and diffuse correlation spectroscopy (DCS) use continuous-wave near-infrared (NIR) point sources and discrete photodetectors to noninvasively measure ScO₂ and CBF respectively in deep brains (up to centimeters). Correspondingly, NIR diffuse optical tomography (DOT) and diffuse correlation tomography (DCT) use dense arrays of sources and detectors to yield boundary measurements across numerous source-detector (S-D) pairs and solve inverse problems to reconstruct 3D images of ScO₂ and CBF, respectively. Tomographic 3D reconstructions by these methods need offline solving of ill-posed nonlinear inverse problems, which are complex and very time consuming. More recently, time-resolved NIRS (TR-NIRS) and time-resolved DCS (TR-DCS) use pulsed point sources and discrete single-photon avalanche diodes to measure temporal point-spread functions and autocorrelation functions for quantifying ScO₂ and CBF in deep brains, respectively. However, most systems suffer from limited numbers of discrete sources and photodetectors, thus taking sparse S-D pair measurements on a limited region-of-interest (ROI) of the head. Expanding the ROI to cover a significant portion of the head introduces great challenges in high-channel-count instrumentation, imaging array ergonomics and data quality management. As a result, their spatial resolutions and head coverages (ROIs) are limited, thus inhibiting the localization of regional brain activations. Moreover, collected cerebral signals are inherently influenced by partial volume artifacts from overlayer tissues of scalp and skull.

### SUMMARY

As such, there is a need for systems and methods for a noninvasive, affordable, and portable optical imaging device with the ability to track blood flow, oxygen levels and TMRO₂ of subjects.

Embodiments of the present disclosure relate to time- and spatially-resolved hyperspectral imaging (TSR-HSI) systems comprising: a wavelength-tunable pulsed laser configured to sequentially emit illumination at a plurality of wavelengths; a coherent pulsed laser having a spectral bandwidth less than 1 nm and configured to generate speckle contrast; a gated single-photon avalanche diode (SPAD) camera configured for time-resolved photon detection across a plurality of temporal gates; a synchronization controller configured to: (i) operate the wavelength-tunable pulsed laser as a master timing source, and (ii) synchronize emission of the coherent pulsed laser and gating of the SPAD camera to the master timing source; and a processor configured to: (a) acquire, using the SPAD camera, both (i) multispectral intensity data from the plurality of wavelengths and (ii) speckle contrast data from the coherent pulsed laser, using the same SPAD camera without spatial co-registration between different imaging sensors, and (b) determine, from the acquired data and across the plurality of temporal gates, depth-resolved values of at least two of: tissue absorption coefficient (μa), tissue scattering coefficient (μ′s), blood flow index (BFI), and oxygen saturation (StO₂), wherein depth resolution is achieved by selecting different temporal gates corresponding to different photon path lengths without performing tomographic reconstruction.

In some embodiments, which may be combined with the above embodiment, the SPAD camera comprises a pixel array of at least 512×512 pixels.

In some embodiments, which may be combined with the above embodiment, each pixel of the pixel array comprises microlenses configured to increase photon detection efficiency by at least a factor of two.

In some embodiments, which may be combined with the above embodiments, the SPAD camera is configured to operate at a sampling rate greater than 20 Hz for acquisition of time-resolved data.

In some embodiments, which may be combined with the above embodiments, the coherent pulsed laser operates at approximately 775 nm and provides a coherence length sufficient to generate diffuse speckle contrast in scattering tissue.

In some embodiments, which may be combined with the above embodiments, wherein the processor is further configured to compute the blood flow index (BFI) as a nonlinear function of speckle contrast and tissue optical properties.

In some embodiments, which may be combined with the above embodiments, the processor is configured to derive metabolic rate of oxygen consumption (TMRO₂) based on both blood flow index (BFI) and oxygen saturation (StO₂).

In some embodiments, which may be combined with the above embodiments, the TSR-HSI system is configured to acquire hyperspectral data over at least 10 wavelengths within a wavelength range of 500 nm to 1000 nm.

In some embodiments, which may be combined with the above embodiments, the TSR-HSI system further comprises optical elements including at least one of: a diffuser, a polarizer, a collimator, or a long-pass filter configured to reduce ambient light and reflection artifacts.

In some embodiments, a mobile imaging system comprises the TSR-HSI system of any of the above embodiments; and a movable platform supporting the wavelength-tunable laser, the coherent pulsed laser, and a control computer.

In some embodiments of the mobile imaging system, which may be combined with the above embodiments, optical components including the SPAD camera and illumination optics are integrated into a probe; and the probe is mounted on an adjustable holder configured for alignment with a region of interest.

Embodiments of the present disclosure also relate to a method for imaging tissue comprising: illuminating the tissue with a sequence of pulsed wavelengths from a wavelength-tunable laser; illuminating the tissue with a coherent pulsed laser configured to produce speckle patterns; synchronizing the wavelength-tunable laser, the coherent pulsed laser, and a gated SPAD camera using a common timing source; acquiring, with the SPAD camera across a plurality of temporal gates, both: (i) multispectral intensity data, and (ii) time-resolved speckle contrast data; selecting different temporal gates to isolate photon paths corresponding to different tissue depths; and computing, from the acquired data, depth-resolved physiological parameters of the tissue, wherein the multispectral intensity data and speckle contrast data are acquired using a single imaging sensor without requiring inter-modality co-registration.

In some embodiments, which may be combined with the above embodiment, selecting different temporal gates provides depth sensitivity of at least 1 mm to 8 mm in biological tissue.

In some embodiments, which may be combined with the above embodiments, the method for imaging tissue further comprises generating a surface geometry map using an isosbestic wavelength and correcting subsequent measurements based on the geometry map.

In some embodiments, which may be combined with the above embodiments, the physiological parameters include at least two of blood flow, oxygenation, and tissue metabolism.

In some embodiments, which may be combined with the above embodiments, a subset of three or fewer wavelengths is used to achieve a sampling rate greater than 10 Hz for monitoring dynamic changes.

Embodiments of the present disclosure also relate to a method for determining tissue optical and physiological parameters comprising: acquiring time-resolved multispectral data at a plurality of wavelengths and temporal gates; fitting the time-resolved multispectral data using a model incorporating: (i) wavelength-dependent absorption, and (ii) wavelength-dependent scattering; simultaneously estimating chromophore concentrations and scattering parameters in a single optimization process using the time-resolved multispectral data; and computing at least one of oxygen saturation (StO₂), blood flow index (BFI), or metabolic rate of oxygen consumption (TMRO₂), wherein the simultaneous estimating avoids a two-step process in which optical properties are first estimated and chromophore concentrations are subsequently derived.

In some embodiments, which may be combined with the above embodiment, scattering is modeled as a power-law function of wavelength characterized by amplitude and exponent parameters.

In some embodiments, which may be combined with the above embodiments, the fitting is performed using a nonlinear least-squares optimization algorithm.

These and other features, aspects, and advantages in accordance with the present disclosure will become better understood with reference to the following drawings, description, and claims.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals.

FIG. 1 illustrates an experimental TR-LSCI setup, according to an exemplary embodiment of the present invention;

FIG. 2A illustrates a conceptual representation of a single wavelength TR-LSCI system to capture 2D flow maps at different depths using different gates, for clarity, only seven gate positions with the interval time of 10 ns (10×1 ns delay time) are illustrated, with the selected offset time of 30.5 ns, shallow and deep depth images were captured at gate positions 4 and 5, respectively;

FIG. 2B illustrates a gate camera intensity profile detected from a calibration tissue phantom at different depths (gates), where the preset parameters include laser frequency: 20 MHz (50 ns); offset time: 0 ns; gate delay time: 1 ns; number of gates: 200; detection time window: 200 ns=200×1 ns, and generally, the offset time ranging from 30 ns to 32 ns was chosen from the shaded region of FIG. 2A, between positions 4 and 5, where longer offset time corresponds deeper imaging depth.

FIG. 2C illustrates a gate camera intensity profile detected from experimental tissue phantoms and animals at different depths (gates) using preset parameters including laser frequency: 20 MHz (50 ns); offset time: 30.5 ns; gate delay time: 18 ps; number of gates: 200; detection time window: 3.6 ns=200×18 ps.

FIG. 3A illustrates an exemplary TR-LSCI setup and for capturing tissue hemodynamics and metabolism maps at different depths (M1 and M2).

FIG. 3B illustrates an exemplary representation of the principle of TR-LSCI for capturing tissue hemodynamics and metabolism maps at different depths (M1 and M2) using different time gates.

FIG. 4A shows the mobile TSR-HSI system, according to an exemplary embodiment of the present invention.

FIG. 4B shows electrical control of the mobile TSR-HSI system, according to an exemplary embodiment of the present invention.

FIG. 5 shows Camera photon detection efficacies (PDEs) with microlenses (μlensed) and without microlenses (non μlensed).

FIG. 6 illustrates experimental results in head-simulating phantoms, where (a)-(b) show top and bottom of a 3D-printed UK logo solid phantom without flow (i.e., flow index=0) with varying top layer thicknesses (1, 2, 3 mm) the empty UK letter channels were filled in liquid phantom solution with Intralipid particle flow (i.e., flow index=1) to generate flow contrasts, where (c)-(f) show reconstructed 2D maps of particle flow contrasts in three phantoms with the top layer thicknesses of 1, 2 and 3 mm respectively, generated by the TR-LSCI at gates #60, #80, #100, and #120 respectively, where the preset parameters for TR-LSCI measurements include laser frequency: 20 MHz (50 ns); offset time: 30.5 ns; gate delay time: 18 ps; number of gates: 200; detection time window: 3.6 ns=200×18 ps, where one hundred (100) time-course images at each gate were taken and averaged to increase SNRs.

FIG. 7 shows TR-LSCI measurements of baseline BFI maps in rodents. FIGS. 7A-7D show the raw intensity image 7A was converted to a height map 7B and a BFI map to extract 3D head surface geometry 7D in a mouse. 7E-7F show 2 D maps of BFI with the intensity and gated (gate #0) modes respectively in a mouse. Spatial resolutions were quantified by selecting two ROIs over a large bundle of superior cerebral veins (L) and a small vessel(S), respectively. 7G-7H show 2D maps of BFI with the gated mode (gate #35) for rat #6 and rat #1, respectively. Spatial resolutions were quantified by selecting two ROIs over the large bundles of superior cerebral veins (L).

FIG. 8A illustrates a raw intensity image of an illustrative rat skull with its scalp retracted.

FIG. 8B illustrates reconstructed BFI maps before, during, and after unilateral and bilateral CCA ligations and releases, followed by CO₂ euthanasia taken at Gate #35 in one illustrative rat, where, for most stages of ligations, ^{~}70 time-course images were averaged to enhance SNRs, while 28 time-course images were averaged during the bilateral ligation phase.

FIG. 8C illustrates a corresponding time-course rCBF changes at two hemispheres (RH and LH) in the same illustrative rat, measured continuously by TR-LSCI with a sampling rate of 0.2 Hz.

FIG. 8D illustrates an average time-course rCBF changes (mean value±standard error) in each hemisphere over 4-6 rats, where the shaded area represents standard errors, and where the preset parameters for the gated mode include laser frequency: 20 MHz (50 ns); offset time: 30.5 ns; gate delay time: 18 ps; number of gates: 80; detection time window: 1.4 ns=200×18 ps.

FIG. 9 shows time-resolved absorptance curves varied with the increase of μₐ and μ′ₛ in intralipid-ink liquid phantoms. FIG. 9A shows μₐ=0.15 cm⁻¹ and μ′ₛ=6.1, 9.8, 15, 20, and 25 cm⁻¹. FIG. 9B shows μₐ=0.06, 0.11, 0.15, 0.21, and 0.26 cm⁻¹ and μ′ₛ=9.8 cm⁻¹.

### DETAILED DESCRIPTION

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

Unless otherwise defined, all terms (including technical and scientific terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the invention, it will be understood that a number of techniques and steps are disclosed. Each of these has individual benefit and each can also be used in conjunction with one or more, or in some cases all, of the other disclosed techniques. Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

In the following description, for purposes of explanation, numerous specific details are set forth in order to provide a thorough understanding of the present invention. It will be evident, however, to one skilled in the art that the present invention may be practiced without these specific details.

The present disclosure is to be considered as an exemplification of the invention and is not intended to limit the invention to the specific embodiments illustrated by the figures or description below.

As is well known to those skilled in the art, many careful considerations and compromises typically must be made when designing for the optimal configuration of a commercial implementation of any system, and in particular, the embodiments of the present invention. A commercial implementation in accordance with the spirit and teachings of the present invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

Unless otherwise indicated, all numbers expressing quantities of ingredients, properties such as reaction conditions, and so forth used in the specification and claims are to be understood as being modified in all instances by the term “about”. Accordingly, unless indicated to the contrary, any numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, the term “subject” refers to a target of administration or medical procedure. The subject of the herein disclosed methods can be a human or animal. The subject may also be a mammal. Thus, the subject of the herein disclosed methods can be a human, nonhuman primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, the term “diagnosed” means having been subjected to a physical examination by a person of skill, for example, a physician, and found to have a condition that can be diagnosed or treated by the compounds, compositions, or methods disclosed herein. For example, “diagnosed with hypoxic-ischemic encephalopathy” means having been subjected to a physical examination by a person of skill, for example, a physician, and found to have a condition that can be described as hypoxic-ischemic encephalopathy.

The present disclosure concerns a time and spatially resolved hyperspectral imaging (TSR-HSI) system. Medical hyperspectral imaging (HSI) is an advanced imaging technique that captures and analyzes light from a wide range of wavelengths, typically beyond what is visible to the human eye. This method provides detailed spectral information about tissues and materials, allowing for enhanced diagnostics and treatment evaluation in various medical applications. Unlike standard imaging, which captures a few wavelengths, HSI collects data across hundreds of wavelengths. This allows for the identification of specific molecular and chemical signatures. By analyzing spectral data from multiple wavelengths, HSI improves the contrast between different tissue types, aiding in the differentiation of healthy versus diseased tissues. Medical spatially resolved hyperspectral imaging (SR-HSI) combines the advantages of HSI with spatial resolution, enabling detailed analysis of tissues at both spatial and spectral levels. Medical SR-HSI uses a wide-field illumination and a 2D camera to acquire 3D data set called hypercube with two spatial dimensions on tissue surface and one spectral dimension, thus providing diagnostic information about tissue physiology, morphology, and composition. HSI/SR-HSI can simultaneously provide multiple pieces of information, such as tissue optical properties, oxygenation, lipid content, water content, and metabolic state. Many systems can provide real-time imaging, allowing for immediate assessment and decision-making during medical procedures. Medical applications of HSI/SR-HSI include tumor detection, wound assessment, surgical guidance, vascular imaging, and disease monitoring. HSI/SR-HSI can differentiate between healthy and diseased tissues based on their spectral signatures. This is particularly useful in oncology for detecting tumors in various organs, including the skin, breast, and brain. In dermatology, hyperspectral imaging helps assess the health of wounds by analyzing the oxygenation levels, aiding in the monitoring of healing processes. Surgeons can use HSI/SR-HSI for enhanced visualization of tissues during procedures, improving the accuracy of tumor resections and reducing damage to surrounding healthy tissues. HSI can visualize blood vessels and assess conditions like vascular malformations or circulatory issues by analyzing the spectral properties of hemoglobin. In chronic conditions such as diabetes, HSI/SR-HSI can monitor tissue changes over time, helping to manage complications related to the disease. However, HSI/SR-HSI has several limitations that can affect its practical application. Key limitations include spatial resolution trade-offs with spectral resolution, motion artifacts, surface sensitivity, small field of view (FOV), scattering effects, calibration challenges, and lighting conditions.

Broadly, embodiments of the present disclosure include a TSR-HSI device and methods of the use thereof. The TSR-HSI device includes one or more diode laser(s), a wavelength-tunable pulsed laser, a coherent pulsed laser set at 775 nm for example, and an image capturing device. In some aspects, the image capturing device is a SPAD (single photon avalanche diode) camera. In some aspects, the wavelength-tunable pulsed laser and/or the coherent pulsed laser may include a diffuser and/or a polarizer and/or a collimator. The image capturing device may include a zoom lens and/or a filter and/or a polarizer. In some aspects, the diode lasers serve to provide alignment optics for a FOV (field of view) while the wavelength-tunable pulsed laser and the coherent pulsed laser provide illumination optics. A computer can then serve as the control hub to synchronize laser pulses from the wavelength-tunable pulsed laser and the coherent pulsed laser and the gated SPAD camera for automated data acquisition. The wavelength-tunable pulsed laser will act as the master, generating preset pulsed triggers (18-82 MHz) to synchronize the two slave devices: the coherent pulsed laser and the gated SPAD camera. A high-speed A/D board (e.g., USB-6451, NI) can then manage the on/off controls of the two pulsed lasers and the gated camera, facilitating sequential data collection. Smaller components, including fiber-coupled pulsed lights, the camera, laser diodes, collimators, diffusers, polarizers, a long-pass NIR filter, and a zoom lens, can then be integrated into a compact probe. This probe can be mounted on a 360° holder for easy alignment to a selected FOV/ROI. Larger components, such as the pulsed lasers, power supplies, and desktop computer, can be mounted on a movable device such as a cart, forming a mobile device. Significantly, the TSR-HSI can utilize a single camera to collect all multispectral data for both BFI and StO₂ measurements, eliminating the complex co-registration typically required in hybrid systems.

The present TSR-HSI system provides the following advanced features over existing HSI/SR-HSI technologies: (1) Innovative TSR-HSI technology for absolute multiparametric measurements of tissue absorption coefficient (μₐ), tissue scattering coefficient (μ′ₛ), blood flow index (BFI), tissue oxygen saturation (StO₂) and tissue metabolic rate of oxygen consumption (TMRO₂). (2) Unique time-resolved technology that enables depth-sensitive imaging of deep tissue properties. (3) Flexibility to tradeoff time, spatial, and spectral resolutions. (4) Fast sampling rate to reduce motion artifacts. (5) Large FOVs. (6) Quantification of scattering effects on tissue hemodynamic measurements/calculations. (7) Acquisition of time-resolved multispectral data by utilizing a single camera, which eliminates the need for complex co-registration typically required in hybrid systems. (8) Adding polarizers and filters to eliminate the influences by source reflection and ambient light. Overall, this cutting-edge TSR-HSI system enables depth-sensitive and high-spatiotemporal imaging of deep tissue optical properties, tissue hemodynamics, and tissue metabolism to enhance the robustness and applicability of hyperspectral imaging in medicine.

The present disclosure in parts concerns the prior development of a time-resolved laser speckle contrast imaging (TR-LSCI) technique that allows for noncontact, continuous 2D mapping of BFI distributions at varying depths over a large FOV over the head of a subject. By employing a time-gated strategy to synchronize pulsed wide-field illumination with a gated SPAD camera, the TR-LSCI approach distinguishes between short and long photon paths through layered head tissues (e.g. scalp, skull, brain), allowing for depth-sensitive quantification of BFI across different head sizes and geometries. Crucially, this method eliminates the need for time-consuming complex 3D tomographic reconstructions, providing real-time 2D mapping of BFI at varying depths into the head.

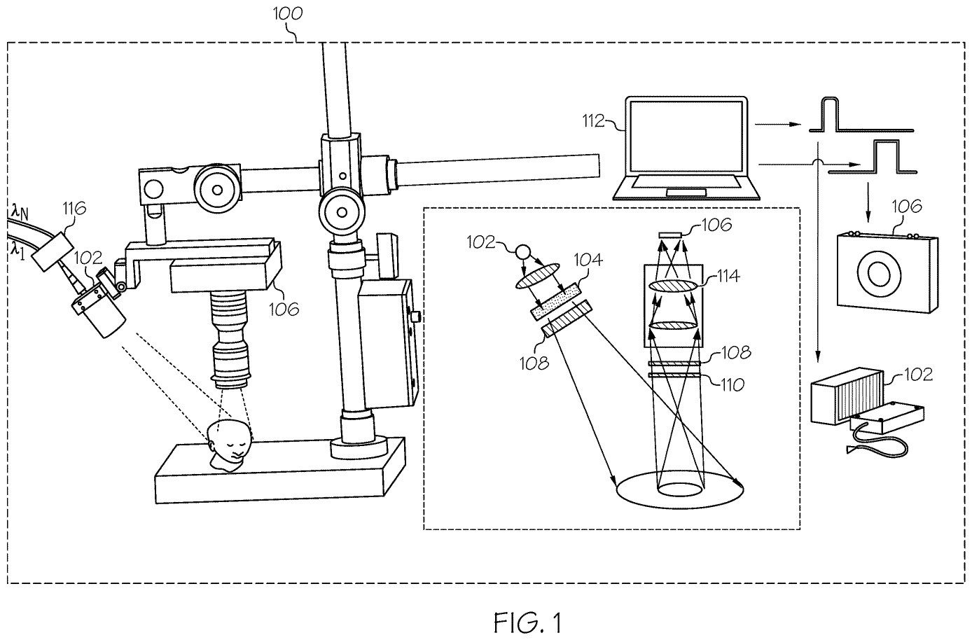

To validate the concept, a benchtop TR-LSCI prototype was assembled as illustrated in FIG. 1, which has been evaluated using head-simulating phantoms and in vivo adult rodents. This prototype has successfully generated multiple BFI maps at various depths, achieving a sampling rate of up to 1 Hz and spatial resolutions ranging from tens to hundreds of micrometers on the tissue surface to several millimeters in deeper brain regions.

### TR-LSCI Prototype

A benchtop single-wavelength TR-LSCI device was developed for measuring tissue BF alone. A free space, picosecond-pulsed, single-mode laser worked as a coherence point source (wavelength: 775 nm; pulse width: 540 ps; spectral bandwidth: <1 nm; max power at 20 MHz: 2 Watt; Katana-08 HP, NKT Photonics). Two engineered optical diffusers (ED1-C20-MD and ED1-S20-MD, Thorlabs) were placed in front of the point source to generate the widefield illumination. The laser power was adjusted so that the maximum photon counts detected by the gated SPAD camera (SwissSPAD2, EPFL, Switzerland) were within its linearity range (<500 counts for 10-bit image). Correspondingly, the power of incident light from the TR-LSCI on the tissue surface was less than 1.2 mW/cm2 (measured by a power meter), which is compliant with the Accessible Emission Limit Class 3R of the American National Standards Institute (ANSI) standard.

SwissSPAD2 is an ultra-high-speed single-photon camera, comprising 512×512 SPAD pixels that can be time-gated. In experiments, a resolution of 256×512 pixels was used to capture speckle contrast images at different depths. SwissSPAD2 achieves a combination of high sensitivity (50% photon detection probability at 520 nm), low pitch (16.38 Rm), moderate fill factor (10.5% fill factor equivalent to 31 μm SPAD active area radius), high temporal resolution (18 ps minimum gate shift), and low dark count rate (0.26 photon counts per second (cps)/μm²). Compared to the majority of other SPAD detectors, the combination of large spatial resolution (large number of pixels) and high temporal resolution (picosecond range) is the greatest advantage of the SwissSPAD2 camera.

A zoom lens (MLM3X-MP, Computar) was coupled to the camera for adjusting ROI size. The F/# of the zoom lens was set at 11 to satisfy the Nyquist criterion based on laser speckle and camera pixel sizes. A pair of polarizers (LPNIRE050-B and LPNIRE200-B, Thorlabs) were added crossing the source and detection paths to reduce the influence of source reflections directly from the tissue surface. A long-pass filter (>750 nm, FEL0750, Thorlabs) was used to minimize the impact of ambient light on TR-LSCI measurements.

FIG. 1 shows a portable TR-LSCI device 100 for measuring BF and StO₂ simultaneously. For illumination of target tissue, at least two pulsed laser sources 102 are turned on alternatively via an optical switch 116 or in free space, each capable of emitting light pulses in nanosecond or picosecond width at NIR range of 600-1100 nm (e.g., λ₁=785 nm and λ₂=830 nm). For detection, a customized SPAD512S camera 106 was used (Pi Imaging Technology) with integrated microlenses and a new operating system (C++) to improve imaging sensitivity, signal-to-noise ratio (SNR) and spatial-temporal resolution. These parts and others (collimator, diffusers 104, polarizers 108, filters 110 and zoom lens 114) can be mechanically integrated into a compact probe fixed on a 360° holder for easy alignment to a selected field of view (FOV)/ROI. Through electrical triggers in the range of 10-80 MHz, a computer 112, as a control unit, synchronizes the pulsed lasers 102 and SPAD512S camera 106 to automatically acquire TR-LSCI data.

The portable TR-LSCI system incorporates at least two nanosecond or picosecond pulsed lasers (λ≥2). The lasers are coupled with a time-gated camera, which can be conveniently housed within a compact probe fixed onto a 360-degree holder for easy alignment with the desired field FOV/ROI. A side illumination configuration can be achieved by positioning the lasers alongside the camera. The pulsed lasers can emit triggers within the range of 10-80 MHz to synchronize their operation with the time-gated camera. The noncontact TR-LCSI system can be placed over any tissue/organ in animals and humans, enabling continuous imaging of BF and StO₂. Typical components of TR-LSCI device include a minimum of two nanosecond or picosecond-pulsed lasers (λ≥2) to generate coherent point light, at least one optical diffuser to provide widefield illumination, at least a pair of polarizers to mitigate source reflections, a zoom lens for adjusting the FOV/ROI, a filter to minimize the influence of ambient light and a time-gated camera for capturing intensity images at varied depths of target tissue.

### TR-LSCI Principle and Data Acquisition

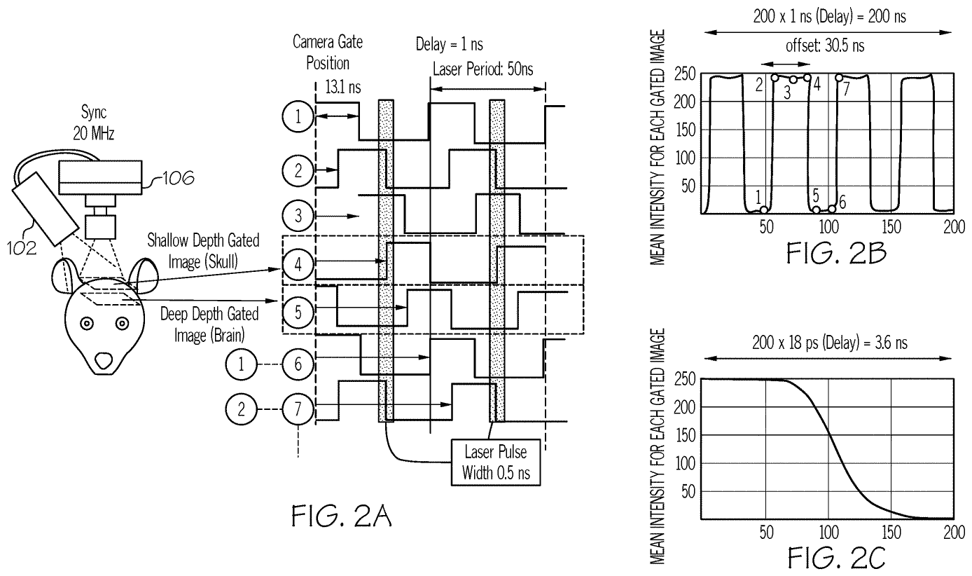

The SwissSPAD2 camera/sensor can operate with two modes: intensity mode and gated mode. The intensity mode, used as the conventional LSCI, operates by having the gate fully open during the exposure window and fully closed during the readout window. In the intensity mode, images are captured with an 8-bit depth, whereas the gated mode allows for capturing images with both 8-bit and 10-bit depths. FIGS. 2A through 2C show the principle and data acquisition of a single-wavelength TR-LSCI with the gated mode. Time gating in SPAD cameras functions as a filter that only captures photons when they arrive within a specific time window. Through electrical triggers produced by the pulsed laser or a signal generator as the master control, the laser and SwissSPAD2 camera are synchronized at 20 MHz (equivalent period of 50 ns= 1/20 MHz) for acquiring multiple gated images at different delay times (minimal 18 picoseconds). Overlapping gates are created when the delay time between adjacent gate windows (also known as gate step) is less than the actual camera gate width (13.1 ns used in this study). Using overlapping gates with a minimal gate delay time of 18 ps, sub-nanosecond temporal resolutions are achieved.

While the pulsed laser illuminates the tissue at 20 MHz, the SwissSPAD2 camera shifts its gated window every delay time (minimal 18 ps) over maximal 200 gate steps, thus collecting photons at different depths inside the target tissue volume (FIG. 2A). Specifically, after acquiring a first gated image at the shallowest depth, the gate shifts one delay time to the next step with respect to the reference from the laser pulse. This is illustrated by the arrows extending from each step, where at step 2, there is a 10× delay; at step 3, there is a 20× delay; at step 4, there is a 30× delay; at step 5, there is a 40× delay; at step 6, there is a 50× delay; and at step 7, there is a 60× delay. Data collections are then repeated over up to 200 gates until the final gate position is reached (i.e., 200th gate at the deepest depth). For illustrative clarity in FIG. 2A, only 7 gate positions within a time interval of 10 ns (10×1 ns delay time) are illustrated instead of the full 200 gates.

Before data acquisition, the offset time is determined to ensure that the first gated image is taken from the tissue surface (i.e., the shallowest depth). The offset is defined as the number of gates to be skipped before useful data acquisition (e.g., from tissue surface) begins. The offset time depends on TR-LSCI instrument configuration such as laser and camera synchronization time, light speed and working distance. To determine the offset time, the SwissSPAD2 camera captured 200 gated images with a delay time of 1 ns over a total of 200 ns (FIG. 2B). Based on the gate intensity profile taken from a calibration tissue phantom, offsets between 30-32 ns (shaded region in FIG. 2A) were used in a typical TR-LSCI setup. For fair comparisons, all experiments in tissue phantoms and animals utilized the constant offset time of 30.5 ns.

After determining the offset time, data acquisition in designed experiments can start with the minimal delay time of 18 ps over 200 gates to maximize depth sensitivity of TR-LSCI measurements (FIG. 2C). As a result, the total detection time window over 200 gates was 3.6 ns (200×18 ps).

To collect one binary frame image at each gate step, the field-programmable gate array (FPGA) implementation in the SwissSPAD2 program involves opening the camera 400 times for data acquisition. The relation between the number of binary frames (Y) and the bit-depth (b) is defined as Y=2^{b}−1. However, since 10-bit images are constructed from four individually saved 8-bit images, Y is equal to 1,020 in 10-bit mode (b=10), instead of 1,023. Therefore, a total of 1020 binary frames are integrated in the FPGA to construct one 10-bit image at each gate step within ^{~}20 ms (1020×400×50 ns). During this acquisition time (^{~}20 ms), the total active exposure time for one 10-bit image is ^{~}5.3 ms (1020×400×13.1 ns). As such, the total time to acquire and save one 10-bit image to a hard drive in the computer is ^{~}30 ms (20 ms for data collection plus 10 ms for data readout and storage).

The frame sampling time of 30 ms is equivalent to a frame rate of ^{~}33 Hz. However, the actual highest frame rate in the experiments was only ^{~}0.2 Hz. The actual SwissSPAD2 frame rates depend on multiple factors including binary frame readout, exposure time (acquisition time) and number of acquired gates. Since the SwissSPAD2 camera operates in global shutter mode, the sensor is insensitive to photons during read-out. Also, data acquisition must be stopped when image data are transferred from the RAM to the hard drive in the computer. Lastly, data acquisition is currently performed through 32-bit MATLAB code, which is not optimal for fast sampling and slower than 64-bit MATLAB. Future optimization of the SwissSPAD2 operating system with new editions of Python code or C++ would contribute to improving the frame rate. Additionally, incorporating a powerful computer equipped with a rapid SSD drive can promote data read and write speeds.

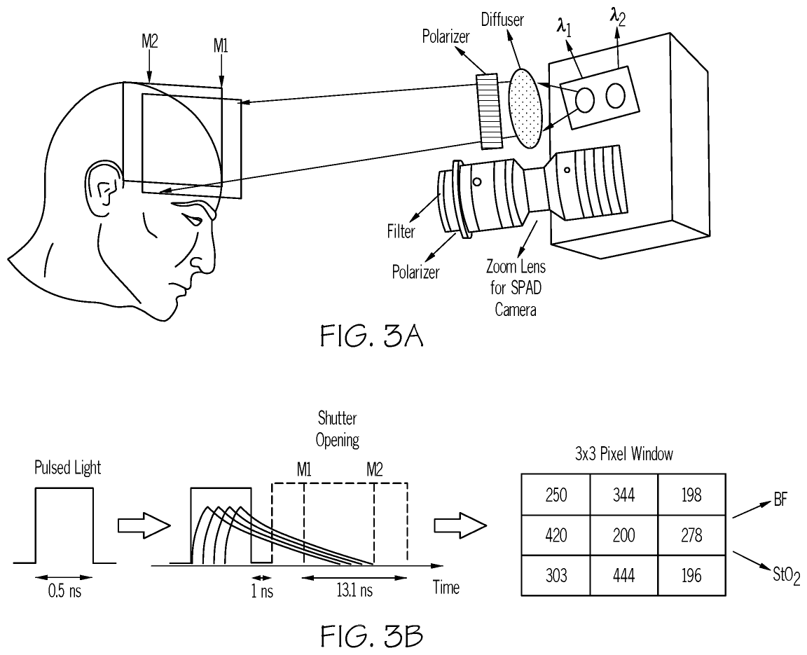

FIGS. 3A and 3B show the principle and data acquisition of TR-LSCI with the gated mode enhancing the depth sensitivity.

The TR-LSCI employs nanosecond or picosecond pulsed NIR lasers at different wavelengths, such as λ₁=785 nm and λ₂=830 nm. Alongside these lasers, a time-gated camera serves as a 2D detector array. This combination enables the simultaneous acquisition of diffuse laser speckle fluctuations for imaging BF and the light intensities (I_{λ})) at multiple wavelengths (λ≥2) for mapping concentrations of oxy-hemoglobin [HbO₂] and deoxy-hemoglobin [Hb], thereby providing a multimodal capability.

Crucially, by implementing time-gated strategies in TR-LSCI measurements, the system, distinguishes between short and long photon paths through the layered head tissues, including scalp, skull and brain. This differentiation allows for quantitatively assessing BF, [HbO₂], and [Hb] noninvasively in different tissue types of both animals and humans, accommodating different tissue size scales and providing multiscale functionality.

For further details regarding the TR-LSCI, pending U.S. patent application Ser. No. 18/473,051 (incorporated herein by reference) may be referenced.

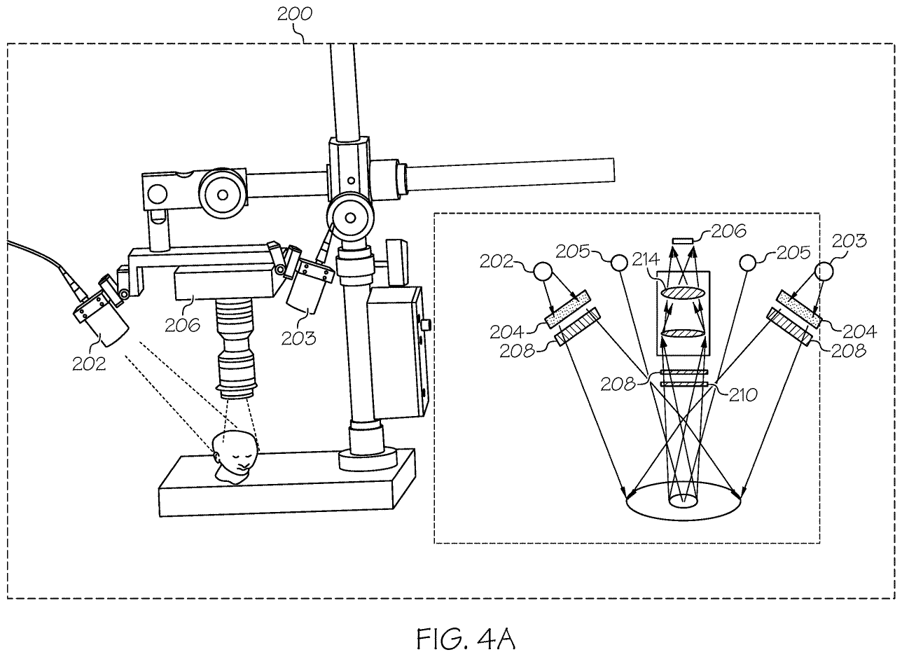

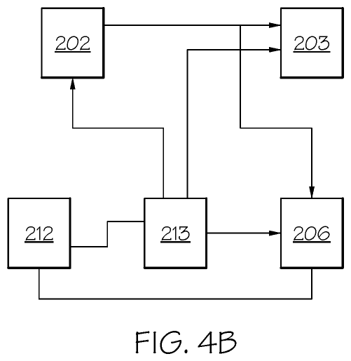

The present disclosure leverages and improves upon the TR-LSCI to develop the time and spatially resolved hyperspectral imaging (TSR-HSI) system. With reference to FIGS. 4A and 4B, an example embodiment of the TSR-HSI system 200 is illustrated. The TSR-HSI system 200 enables depth-sensitive and high-spatiotemporal imaging of tissue absorption coefficient μₐ, tissue scattering coefficient μ′ₛ, tissue oxygenation saturation StO₂, tissue blood flow index (BFI), and metabolic rate of tissue oxygen consumption (TMRO₂).

Illumination System with Pulsed Lasers

A wavelength-tunable laser pulsed laser 202 (e.g., SuperK FIANIUM FIR-20, NKT Photonics; Chameleon Ultra II, Coherent; Mai Tai HP, Spectra-Physics) with fiber-coupled output may be integrated into the system for StO₂ measurements. Given that tunable lasers usually have a wide spectral width (>2 nm), they may not provide adequate coherence length for capturing diffuse speckle contrasts (essential for BFI signals). To address this, the TSR-HSI system 200 additionally couples light from the coherent pulsed laser 203 at a single wavelength. For example, the coherent pulsed laser 203 may generate a single wavelength at 775 nm with a spectral bandwidth of <1 nm (KATANA-08 HP, NKT Photonics) for BFI measurements. Additionally, two green laser diodes 205 may be used to ensure a preset working distance by aligning the two beams to a common spot on the target tissue.

Detection System with Customized High-Performance SPAD Camera

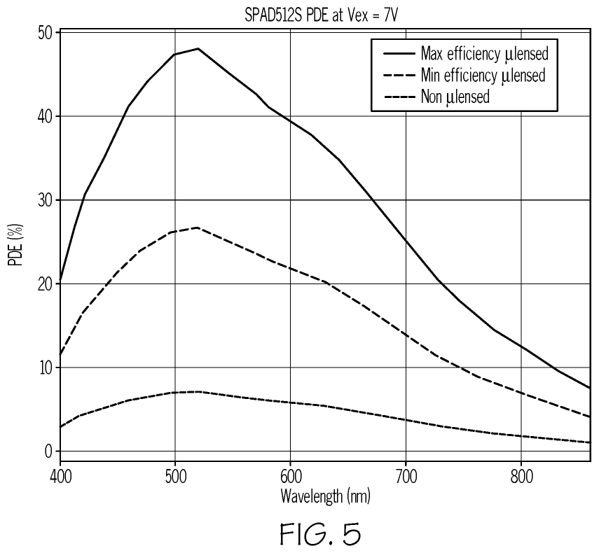

The SwissSPAD2 camera was used in the initial TR-LSCI prototypes for depth-sensitivity imaging of BFI. To enhance the performance in the TSR-HSI system 200, a SPAD512S camera was leveraged as the gated single-photon avalanche diode camera 206. The SPAD512S includes (a) Enhanced Photon Collection, (b) Higher Spatial Resolution, (c) Increased Sampling Rate, and (d) Compact and User-Friendly Design. The SPAD512S integrates 512×512 microlenses on the sensor pixels, significantly increasing the effective fill factor from 10% to 50-90%. This enhancement is expected to boost the photon detection efficiency (PDE) by 4-6 times as illustrated in FIG. 5, leading to an improvement in signal-to-noise ratio (SNR) by 2-3 times, and enabling maximal imaging depth of ≥8 mm. SPAD512S offers improved spatial resolution with 512×512 pixels, compared to the 256×512 pixels of SwissSPAD2. This upgrade will allow for more detailed imaging of tissue vasculature. The new operating system (C++) will enhance the camera's sampling rate from 1 Hz (MATLAB) to more than 50 Hz, allowing for more rapid data acquisition and improved temporal resolution. Finally, the SPAD512S camera is designed to be compact (and easy to operate, facilitating its use in various settings and by users with varying levels of technical expertise.

### System Control and Data Acquisition

With reference to FIG. 4B, a desktop computer comprising a processor 212 may serve as the control hub, synchronizing the wavelength tunable pulsed laser 202, the coherent pulsed laser 203, and the SPAD camera 206 for automated data acquisition. The wavelength-tunable pulsed laser 202 may act as the master, generating preset pulsed triggers (18-82 MHz) to synchronize the two slave devices: the coherent pulsed laser 203 at 775 nm and the SPAD camera 206. A high-speed analog-to-digital (A/D) board 213 (e.g., USB-6451, NI) may manage the on/off controls of the wavelength tunable pulsed laser 202, the coherent pulsed laser 203, and the SPAD camera 206, facilitating sequential data collection. For imaging StO₂, the TSR-HSI system 200 will collect multispectral intensity data (Iλ₁, Iλ₂, . . . Iλₙ) by scanning multiple wavelengths (λ₁ to λₙ) generated by the wavelength tunable pulsed laser 202. For imaging BFI, the TSR-HSI system 200 will capture diffuse speckle contrast data (K_{S}) generated by the coherent pulsed laser 203 at 775 nm.

### Compact Probe Design and Mobile Device Assembly

The various smaller components, including the SPAD camera 206, laser diodes 205, collimator, diffusers 104, polarizers 108, filters 110 and zoom lens 114, may be integrated into a compact probe. This probe may be mounted on a 360° holder for easy alignment to a selected FOV/ROI. Larger components, such as the wavelength tunable pulsed laser 202, the coherent pulsed laser 203, power supplies, and desktop computer with processor 212, may be mounted on a movable cart, forming a mobile device. Importantly, the TSR-HSI system 200 employs a single SPAD camera 206 to collect all multispectral data for both BFI and StO₂ measurements, eliminating the complex co-registration typically required in hybrid systems.

### User-friendly Operating System With Fast Sampling Rate

The operating system can be optimized to be “plug-and-play” so that a neuroscientist can easily use it without specialized training. The control panel of the computing device can have only a few buttons to control the device (e.g., start, marking events, stop), input boxes to set up parameters (e.g., ROI, camera pixel-window size, gated time, exposure time, and frame rate) and display windows to automatically report resulting images. For example, in one or more embodiments, a user-friendly GUI is generated in C++ to control the TSR-HSI system 200. The GUI may feature a limited number of buttons, input boxes, and display windows. The buttons may allow users to control the TSR-HSI system 200 for essential functions, such as: “start”, “marking events”, and “stop”. Several input boxes may allow users to configure key parameters, including ROI/FOV, laser pulse rate, laser power, number of wavelengths (λₙ), wavelength bandwidth, wavelength range, camera offset time, camera gate number, and camera exposure time. Further, multiple display windows may automatically report resulting images and relevant data in real time.

The total data acquisition time is dependent on the preset number of scanning wavelengths (n). For hyperspectral measurements of absolute baseline values (e.g., scanning 100 wavelengths in the range of 500-1000 nm for StO₂, plus one wavelength at 775 nm for BFI) the minimal sampling time is approximately 2s: 101 wavelengths×(100 μs wavelength switching time+20 ms camera acquisition time). For multispectral measurements of absolute baseline values (e.g., scanning 16 wavelengths in the range of 500-1000 nm for StO₂, plus one wavelength at 775 nm for BFI), the minimal sampling time is approximately 342 ms: 17 wavelengths×(100 μs wavelength switching time+20 ms camera acquisition time). For three-wavelength measurements of fast relevant changes (e.g., 690 nm and 830 nm for StO₂, plus 775 nm for BFI), the minimal sampling time is approximately 60.3 ms: 3 wavelengths×(100 μs wavelength switching time+20 ms camera acquisition time). Accordingly, the maximum sampling rate for acquiring a full frame of StO₂ and CBF images will vary as follows: 0.5 Hz for 101 wavelengths, 2.9 Hz for 17 wavelengths, and 16.6 Hz for 3 wavelengths.

The following methods and algorithms will be explored to fit multispectral images obtained by the TSR-HSI at varying gates, enabling the reconstruction of 3D images that represent tissue hemodynamics and metabolism.

Fitting Multispectral Data for Imaging StO₂. Conventional HSI methods involve in two steps: pre-processing and spectral unmixing. The purpose of pre-processing is to remove the spectral non-uniformity of the illumination device and the influence of dark current. The white reference spectral cube I(x, y, λᵢ) at each pixel (x, y) is acquired by spectral imaging of a standard white reference phantom. The dark reference cube I_{dark} (x, y, λᵢ) is captured by keeping the camera shutter closed. Hyperspatial raw dataset (x, y, λᵢ) is converted into the absorption

$$A ( x , y , {λ}_{i} ) = - {log}_{10} \frac{I ( x , y , {λ}_{i} ) - {I}_{dark} ( x , y , {λ}_{i} )}{{I}_{white} ( x , y , {λ}_{i} ) - {I}_{dark} ( x , y , {λ}_{i} )} \qquad ( Eq . 1 )$$

For simplicity, in accordance with the present disclosure two chromophores (oxyhemoglobin HbO₂ and deoxyhemoglobin Hb) absorption model may be used for unmixing hyperspectral dataset. According to Beer-Lambert law, the absorbance can be estimated.

A(x,y,λᵢ)=ε_{oxy}(λᵢ)C_{oxy}(x,y)L+ε_{deoxy}(λᵢ)C_{deoxy}(x,y)L+G (Eq. 2)

Where C(x,y) is a chromophore concentration at the coordinate (x,y) and ε(λᵢ) is a spectral molar extinction coefficient. The “oxy” and “deoxy” refer to the HbO₂ and Hb, which are the primary chromophores responsible for the absorption of NIR light. The mean-free path L is the average distance traveled by a photon within the tissue. The term G accounts for the light scattering effect. The ε_{deoxy}(λᵢ) and ε_{oxy} (λᵢ) can be found in the literature. The unknowns L and G are assumed to be wavelength independent. Based on the linear model, the unknown variables of C_{oxy} (x,y), C_{deoxy} (x,y), L, and G can be fitted simultaneously by minimizing the term:

$${𝒳}^{2} = {\sum }_{i = 1}^{n} {\vert {A}_{λ i}^{m e a s u r e d} - {A}_{λ i}^{c a l c u l a t e d} \vert}^{2} \qquad ( Eq . 3 )$$

StO₂ is computed by:

$${StO}_{2} = \frac{{C}_{oxy} ( x , y )}{{C}_{oxy} ( x , y ) + {C}_{deoxy} ( x , y )} \qquad ( Eq . 4 )$$

Note that additional unknowns may be included in Eq. 2 to extract water content, lipid concentration, and the oxidation state of cytochrome c oxidase (oxCCO).

Fitting time-resolved multispectral data for imaging μₐ (λ), μ′ₛ (λ) and StO₂. Conventional methods for estimation of chromophore concentrations from time-resolved spectral data are based on a two-step procedure⁷. In the first step optical parameters μₐ (λ) and μ′ₛ(λ)) are calculated by fitting time-resolved curves to an analytical or numerical solution of transport or diffusion equation. In the second step the absorption spectrum is exploited to calculate the chromophore concentrations (Eq. 2) and StO₂ (Eq. 4). Analyzing the time-resolved curves at different wavelengths in a single step for direct assessment of μₐ (λ), μ′ₛ (λ), and StO₂ has been shown to be more robust than what the conventional two-step produces, especially at a low SNR. The time-resolved absorption curve may be expressed as:

A(t,λ;μₐ,μ′ₛ)=S(t,μ′ₛ)exp(−μₐvt) Eq. 5

where is the speed of light in the tissue. By replacing μₐ(λ)=ΣᵢCᵢεᵢ (λ) and μ′ₛ=a(λ/λ₀) in Eq. 5, the equation becomes:

A(t,λ;a,b·Cᵢ)=S(t,a(λ/λ₀)^{-b})exp(−vtΣᵢCᵢεᵢ(λ)) Eq. 6,

Where λ₀ is a reference wavelength, a is scatter amplitude, and b is scatter power. To calculate unknowns (C_{oxy} and C_{deoxy}, a, and b) the fitting is carried out by minimizing the difference between the experimental and theoretical vectors of all the time resolved curves using a standard Levenberg-Marquadt algorithm. Finally, μₐ (λ) and μ′ₛ(λ) are calculated by Eq. 6 and StO₂ is computed by Eq. 4.

Input μₐ(λ), μ′ₛ(λ) to a nonlinear function for quantifying BFI. The diffuse speckle contrast Kₛ has been established as a nonlinear function of tissue optical properties and BFI. Kₛ (λ)=f(aD_{B},μₐ(λ),μ′ₛ(λ)T,β) Eq. 7, where aD_{B} is the BFI, T is the camera exposure time, and β (coherence factor) is a constant determined by the geometry and collection optics of the experiment. The μₐ(λ), μ′ₛ(λ) obtained by the methods herein may be input to Eq. 7 for quantifying absolute BFI (aD_{B}).

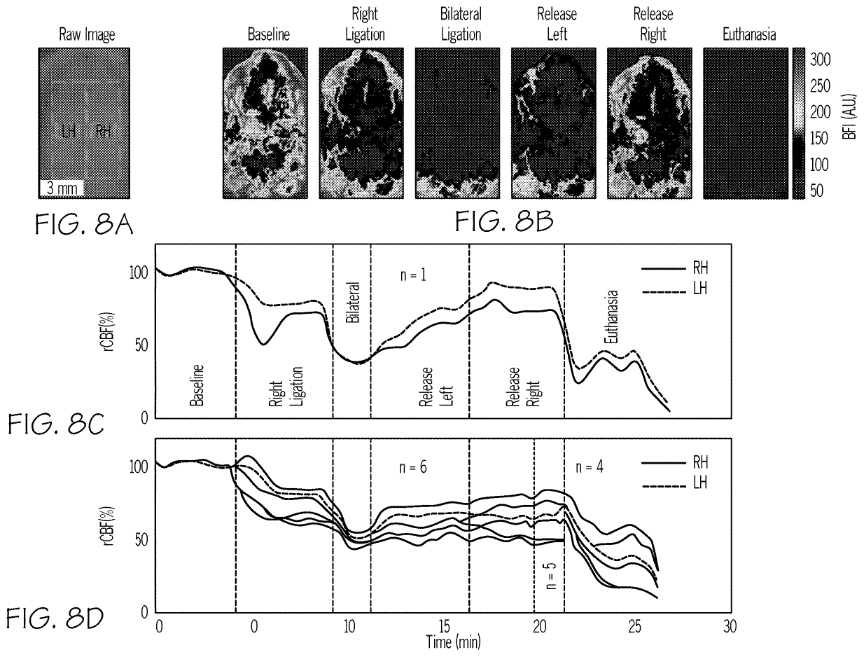

Capture Fast Relative Changes in rBFI, ΔStO₂, and rTMRO₂ using limited number of wavelengths. A limited number of wavelengths (e.g., three wavelengths: 690, 775, 830 nm) at sampling rates up to 16.6 Hz may be used to capture fast changes in rBFI, ΔStO₂, and rTMRO₂. rBFI at each pixel may be obtained by use of the coherent pulsed laser 203 at 775 nm and calculated by normalizing BFI changes to its baseline (see FIG. 8). ΔStO₂ at each pixel will be obtained by use of two wavelengths (690 and 830 nm) and calculated based on the Beer-Lambert law. rTMRO₂ at each pixel will be calculated based on Fick's law: rTMRO₂=rBFI×(SaO₂−StO₂)/(SaO_{2baseline}−StO_{2baseline}), where StO₂ and StO₂ baseline are time-course dynamic change and baseline value respectively, measured by the TSR-HSI. SaO₂ and SaO₂ baseline are measured by a Respirator-Oximeter (Smiths Medical). Finally, absolute changes in these quantities will be obtained by multiplying their absolute baseline values obtained by methods herein.

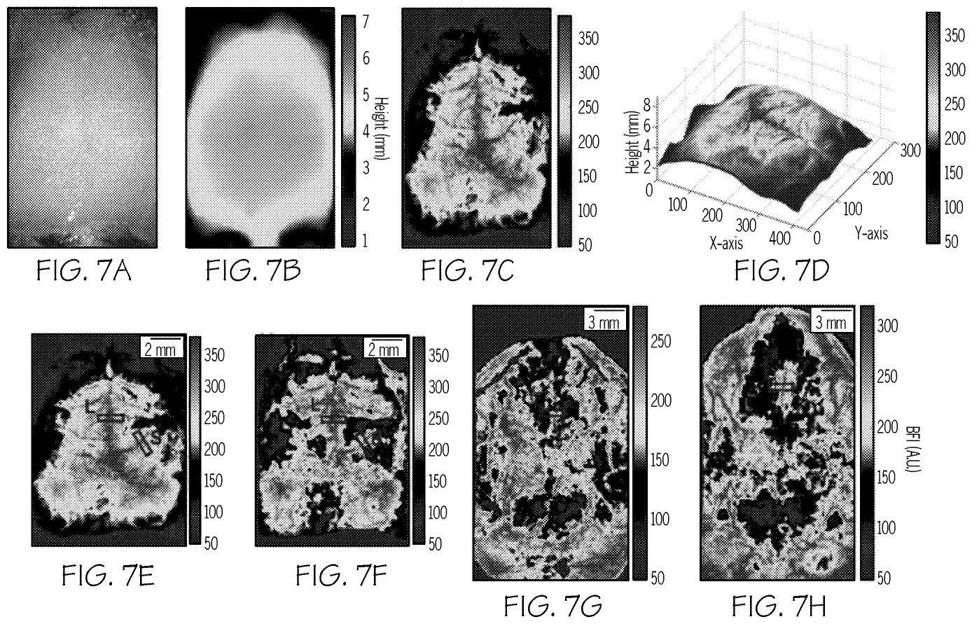

Correct Head Geometry Influence on Tissue Hemodynamic Measurements. To minimize the impact of irregular head geometry on the measured absorbance (x, y, λᵢ) as defined in Eq. 1, the pulsed laser is first tuned to a wavelength at an isosbestic point, such as 500 nm, where the absorption by HbO₂ and Hb is equal. This will allow us to obtain a height map of the head surface geometry under intensity mode as illustrated in FIG. 7D. Following the initial geometry mapping, the TSR-HSI system 200 will conduct multispectral measurements across the NIR range (e.g., 650 to 950 nm) using gated mode for depth-sensitive imaging. During these measurements, the working distance between the light source and the head will be fixed to reduce variations in detected absorptance. Finally, the multispectral raw dataset (x, y, λᵢ) will be corrected by subtracting the reference dataset I(x, y, λ₅₀₀ nm). This subtraction will account for the influence of head geometry on the detected light intensity, allowing for more accurate assessments of the underlying hemodynamic parameters.

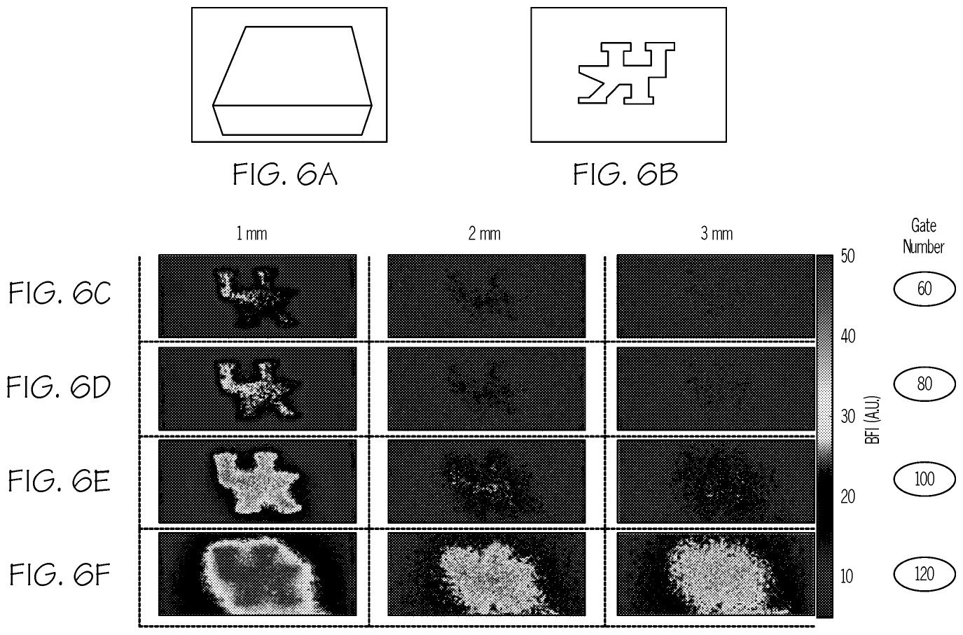

TR-LSCI Enables Depth-sensitivity Detection of Flow Contrasts in Head-simulating Phantoms. With reference to FIG. 6, three solid phantoms, featuring empty channels that display the University of Kentucky logo (“UK”), were designed and fabricated with varying top layer thicknesses of 1, 2, and 3 mm to mimic small animal heads with different skull structures. These solid phantoms were composed of resin, India ink, and titanium dioxide (TiO₂), with the UK logo channels filled with a liquid phantom made of water, Intralipid particles, and India ink. The concentration of India ink regulated the tissue absorption coefficient (μₐ), while TiO₂ and Intralipid concentrations controlled the tissue reduced scattering coefficient (μ′ₛ). The liquid phantom also simulated the Brownian motion of Intralipid particles to mimic red blood cell movement in the brain (i.e., CBF).

The optical properties of both solid and liquid phantoms were established at μₐ=0.03 cm⁻¹ and μ′ₛ=9 cm⁻¹. The intensity images captured by the SwissSPAD2 across different gates were converted into diffuse laser speckle contrast images representing various depths (FIG. 6c-6f). The diffuse speckle contrast for each gate was quantified by calculating the intensity ratio of standard deviation (σσ) to mean (<I>) within a small pixel window (e.g., 3×3 pixels), defined as K_{S}=σ/<I>. While the relationship between K_{S} and BFI is nonlinear, it can be approximated as

$$\frac{1}{{K}_{s}^{2}} .$$

This simplification allows for rapid processing of K_{S} values across all pixels within the selected region-of-interest (ROI), generating a BFI map. A developed MATLAB-based algorithm, leveraging the Parallel Computing Toolbox and Image Processing Toolbox, enables real-time calculation of BFI maps (FIG. 6c-6f). Notably, higher flow contrasts corresponding to the UK logo were observed at greater depths with larger gate numbers, confirming the depth sensitivity of TR-LSCI. Additionally, the signal-to-noise ratio (SNR) diminished with increased imaging depth (from gate #60 to #120) and top layer thickness (from 1 to 3 mm) due to fewer detected photons.

Additionally, the maximum penetration depth of TR-LSCI using a two-layer solid/liquid phantom setup was assessed. A solid slab phantom was placed over an Intralipid liquid phantom, which was heated to induce temperature variations. The TR-LSCI successfully detected particle flow changes with the temperature variations through a 5 mm thick solid slab, showcasing its potential for monitoring flow dynamics beneath top layers.

TR-LSCI Captures Cerebral Vasculature and Head Geometry (FIG. 7). FIGS. 7a-7d illustrate the process of obtaining various TR-LSCI imaging outputs, including the raw intensity image, intensity height map, BFI map, and a 3D presentation of the BFI overlaying the height map in a mouse. The height map, generated from the intensity image, provides a detailed representation of head surface geometry (FIG. 7d). FIGS. 7e-7f display BFI maps obtained from one mouse, showcasing both intensity and gated modes. In contrast, FIGS. 7g-7h present BFI maps from two rats utilizing the gated mode. To assess spatial resolutions, different ROIs over identifiable vessels of varying sizes were selected. Analysis of the pixel counts within these ROIs, relative to the head scales, revealed that the spatial resolutions of TR-LSCI range from tens to hundreds of micrometers. It is important to note that the visibility of large vessel bundles in the rats is partially limited due to the partial volume effects caused by their thicker skulls, which can obscure fine details of the underlying cerebral vasculature.

TR-LSCI Captures CBF Variations During Transient Carotid Artery Ligations in Rats (FIG. 8). FIGS. 8a-8c illustrate the setup and results for an illustrative rat (Rat #1) subjected to transient bilateral ligation of the common carotid artery (CCA). The images include the raw intensity map, BFI maps, and corresponding time-course relative cerebral blood flow (rCBF) changes. Following a baseline measurement of 5 minutes, the right CCA was occluded by tightening the suture for 5 minutes. Subsequently, the left CCA was also ligated to induce bilateral occlusion for an additional 2 minutes. The sequential release of the ligations for both the left and right CCAs occurred over the next 5 minutes each, allowing for monitoring of CBF recovery from transient global ischemia. FIG. 8d presents the average results from a group of 6 rats, showcasing the significant variations in rCBF corresponding to the hemispheres during the ligation and release phases. This variability demonstrates the effectiveness of TR-LSCI in detecting disruptions in rCBF. Notably, at the conclusion of the euthanasia process, the rCBF approached “zero,” aligning with physiological expectations for the cessation of CBF.

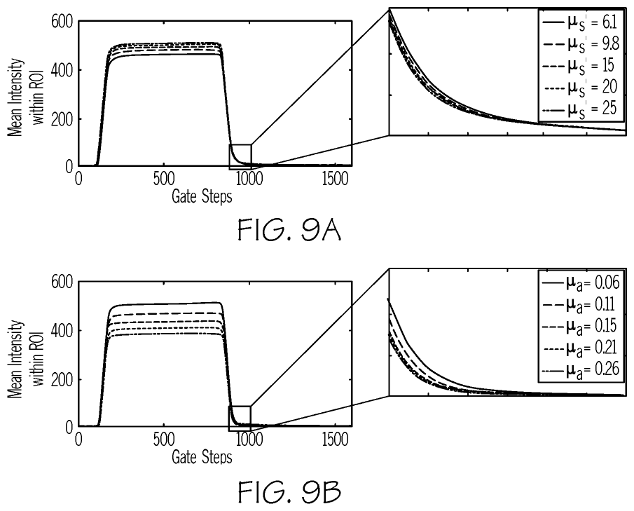

For proof of concept, tissue phantom titration experiments were conducted to evaluate the impacts of μₐ and μ′ₛ, on time/gate resolved absorptances detected (FIG. 9). Step increases in μμdd and μμ′ were created by adding Indian inks and Intralipid solution, respectively. The commercial Imagent (ISS) was utilized to confirm the μₐ and μ′ₛ values at each step. From the TR-LSCI measurements, with reference to FIG. 9B, the expected intensity reductions and profile shifts of time/gate resolved curves due to the increases of μ′ₛ (FIG. 7a) and μₐ were observed, respectively. To adapt the gated data to Eq. 5-6, the time t was replaced with the gate steps, assuming a homogenous tissue along the photon pathway. The depth sensitivity is maintained by selecting different initial gate numbers. A larger initial gate number corresponds to a deeper depth.

It should be apparent to those skilled in the art that various modifications and variations may be made to the embodiments described within without departing from the spirit and scope of the claimed subject matter. Thus, it is intended that the specification cover the modifications and variations of the various embodiments described within provided such modification and variations come within the scope of the appended claims and their equivalents.

As used throughout, the singular forms “a,” “an” and “the” include plural referents unless the context clearly dictates otherwise. Thus, for example, reference to “a” component includes aspects having two or more such components, unless the context clearly indicates otherwise.

Optional or optionally means that the subsequently described event or circumstances may or may not occur. The description includes instances where the event or circumstance occurs and instances where it does not occur.

It should be understood that any two quantitative values assigned to a property or measurement may constitute a range of that property or measurement, and all combinations of ranges formed from all stated quantitative values of a given property or measurement are contemplated in this disclosure.

As used here and in the appended claims, the words “comprise,” “has,” and “include” and all grammatical variations thereof are each intended to have an open, non-limiting meaning that does not exclude additional elements or steps.

Having described the subject matter of the present disclosure in detail and by reference to specific embodiments thereof, it is noted that the various details disclosed within should not be taken to imply that these details relate to elements that are essential components of the various embodiments described within, even in cases where a particular element is illustrated in each of the drawings that accompany the present description. Further, it should be apparent that modifications and variations are possible without departing from the scope of the present disclosure, including, but not limited to, embodiments defined in the appended claims. More specifically, although some aspects of the present disclosure are identified as particularly advantageous, it is contemplated that the present disclosure is not necessarily limited to these aspects. In particular, it should be understood that while specific embodiments of the temperature oscillation overlay device have been described with reference to particular hardware configurations, wireless protocols, temperature sensor types, and biological applications, the present disclosure is not limited to these specific implementations, and that equivalent structures, methods, and applications that achieve the same functional result of superimposing a programmable biologically defined time-varying temperature profile onto an existing tissue culture incubator without permanent modification thereof are contemplated as falling within the scope of the present disclosure.

What is claimed is:

1. A time- and spatially-resolved hyperspectral imaging (TSR-HSI) system comprising:
a wavelength-tunable pulsed laser configured to sequentially emit illumination at a plurality of wavelengths;
a coherent pulsed laser having a spectral bandwidth less than 1 nm and configured to generate speckle contrast;
a gated single-photon avalanche diode (SPAD) camera configured for time-resolved photon detection across a plurality of temporal gates;
a synchronization controller configured to:
(i) operate the wavelength-tunable pulsed laser as a master timing source, and
(ii) synchronize emission of the coherent pulsed laser and gating of the SPAD camera to the master timing source; and
a processor configured to:
(a) acquire, using the SPAD camera, both (i) multispectral intensity data from the plurality of wavelengths and (ii) speckle contrast data from the coherent pulsed laser, using the same SPAD camera without spatial co-registration between different imaging sensors, and
(b) determine, from the acquired data and across the plurality of temporal gates, depth-resolved values of at least two of: tissue absorption coefficient (μa), tissue scattering coefficient (μ′s), blood flow index (BFI), and oxygen saturation (StO₂),
wherein depth resolution is achieved by selecting different temporal gates corresponding to different photon path lengths without performing tomographic reconstruction.

2. The system of claim 1, wherein the SPAD camera comprises a pixel array of at least 512×512 pixels.

3. The system of claim 2, wherein each pixel of the pixel array comprises microlenses configured to increase photon detection efficiency by at least a factor of two.

4. The system of claim 1, wherein the SPAD camera is configured to operate at a sampling rate greater than 20 Hz for acquisition of time-resolved data.

5. The system of claim 1, wherein the coherent pulsed laser operates at approximately 775 nm and provides a coherence length sufficient to generate diffuse speckle contrast in scattering tissue.

6. The system of claim 1, wherein the processor is further configured to compute the blood flow index (BFI) as a nonlinear function of speckle contrast and tissue optical properties.

7. The system of claim 1, wherein the processor is configured to derive metabolic rate of oxygen consumption (TMRO₂) based on both blood flow index (BFI) and oxygen saturation (StO₂).

8. The system of claim 1, wherein the system is configured to acquire hyperspectral data over at least 10 wavelengths within a wavelength range of 500 nm to 1000 nm.

9. The system of claim 1, further comprising optical elements including at least one of: a diffuser, a polarizer, a collimator, or a long-pass filter configured to reduce ambient light and reflection artifacts.

10. A mobile imaging system comprising:
the system of claim 1; and
a movable platform supporting the wavelength-tunable laser, the coherent pulsed laser, and a control computer.

11. The system of claim 10, wherein:
optical components including the SPAD camera and illumination optics are integrated into a probe; and
the probe is mounted on an adjustable holder configured for alignment with a region of interest.

12. A method for imaging tissue, comprising:
illuminating the tissue with a sequence of pulsed wavelengths from a wavelength-tunable laser;
illuminating the tissue with a coherent pulsed laser configured to produce speckle patterns;
synchronizing the wavelength-tunable laser, the coherent pulsed laser, and a gated SPAD camera using a common timing source;
acquiring, with the SPAD camera across a plurality of temporal gates, both:
(i) multispectral intensity data, and
(ii) time-resolved speckle contrast data;
selecting different temporal gates to isolate photon paths corresponding to different tissue depths; and
computing, from the acquired data, depth-resolved physiological parameters of the tissue,
wherein the multispectral intensity data and speckle contrast data are acquired using a single imaging sensor without requiring inter-modality co-registration.

13. The method of claim 12, wherein selecting different temporal gates provides depth sensitivity of at least 1 mm to 8 mm in biological tissue.

14. The method of claim 12, further comprising generating a surface geometry map using an isosbestic wavelength and correcting subsequent measurements based on the geometry map.

15. The method of claim 12, wherein the physiological parameters include at least two of blood flow, oxygenation, and tissue metabolism.

16. The method of claim 12, wherein a subset of three or fewer wavelengths is used to achieve a sampling rate greater than 10 Hz for monitoring dynamic changes.

17. A method for determining tissue optical and physiological parameters, comprising:
acquiring time-resolved multispectral data at a plurality of wavelengths and temporal gates;
fitting the time-resolved multispectral data using a model incorporating:
(i) wavelength-dependent absorption, and
(ii) wavelength-dependent scattering;
simultaneously estimating chromophore concentrations and scattering parameters in a single optimization process using the time-resolved multispectral data; and
computing at least one of oxygen saturation (StO₂), blood flow index (BFI), or metabolic rate of oxygen consumption (TMRO₂),
wherein the simultaneous estimating avoids a two-step process in which optical properties are first estimated and chromophore concentrations are subsequently derived.

18. The method of claim 17, wherein scattering is modeled as a power-law function of wavelength characterized by amplitude and exponent parameters.

19. The method of claim 17, wherein the fitting is performed using a nonlinear least-squares optimization algorithm.
